(19) United States
(12) Patent Application Publication
(10) Pub. No.: US 2012/0071983 A1
(43) Pub. Date: Mar. 22, 2012

(54) BONE GRAFTS

(76) Inventors: Eddie F. Ray, III, Cordova, TN (US); Lawrence M. Boyd, Memphis, TN (US); James E. Van Hoeck, Cordova, TN (US); Bradley J. Coates, Rossville, TN (US); Jeffrey W. Poyner, Atoka, TN (US)

(21) Appl. No.: 13/237,711

(22) Filed: Sep. 20, 2011

Related U.S. Application Data
(63) Continuation of application No. 12/458,653, filed on Jul. 20, 2009, now abandoned.
Application No. 12/458,653 is a continuation of application No. 10/766,504, filed on Jan. 27, 2004, now abandoned.
Application No. 10/766,504 is a continuation-in-part of application No. 10/114,675, filed on Apr. 2, 2002.
Application No. 10/114,675 is a continuation of application No. 09/484,354, filed on Jan. 18, 2000, now Pat. No. 6,371,988.
Application No. 09/484,354 is a division of application No. 08/740,031, filed on Oct. 23, 1996, now abandoned.
Application No. 10/766,504 is a continuation-in-part of application No. 09/448,086, filed on Nov. 23, 1999, now Pat. No. 7,276,081.
Application No. 09/448,086 is a continuation of application No. 08/948,135, filed on Oct. 9, 1997, now Pat. No. 5,989,289.
Application No. 08/948,135 is a division of application No. 08/902,937, filed on Jul. 30, 1997, now abandoned.

(51) Int. Cl. A61F 2/44

(52) U.S. Cl. 623/17.16

(57) ABSTRACT

Spinal spacers for fusion of a motion segment. A spinal spacer includes a body, which may be formed of a bone composition. The body includes a first end, an opposite second end, a superior face defining a superior vertebral engaging surface and an inferior face defining an inferior vertebral engaging surface. At least one of the vertebral engaging surfaces defines a set of migration resistance grooves. Each of the grooves includes a first face defining an angle of no more than about 90 degrees relative to the engaging surface and a second opposing sloped face.

This application is a continuation-in-part of U.S. patent application Ser. No. 10/114,675, filed Apr. 2, 2002, which was a continuation of U.S. patent application Ser. No. 09/484,354, filed Jan. 18, 2000 (now U.S. Pat. No. 6,371,988, issued Apr. 16, 2002), which was a division of U.S. patent application Ser. No. 08/740,031, filed Oct. 23, 1996, now abandoned.

This application is also a continuation-in-part of U.S. patent application Ser. No. 09/448,086, filed Nov. 23, 1999, which was a continuation of U.S. patent application Ser. No. 08/948,135, filed Oct. 9, 1997 (now U.S. Pat. No. 5,989,289, issued Nov. 23, 1999), which was a continuation of U.S. patent application Ser. No. 08/902,937, filed Jul. 30, 1997, now abandoned.

The entirety of each of the noted U.S. patents and patent applications is incorporated herein by reference.

### FIELD OF THE INVENTION

The present invention relates to spacers, compositions and methods for arthrodesis. In specific applications of the invention the spacers include bone grafts in synergistic combination with osteogenic compositions.

### BACKGROUND OF THE INVENTION

Spinal fusion is indicated to provide stabilization of the spinal column for painful spinal motion and disorders such as structural deformity, traumatic instability, degenerative instability, and post-resection iatrogenic instability. Fusion, or arthrodesis, is achieved by the formation of an osseous bridge between adjacent motion segments. This can be accomplished within the disc space, anteriorly between contiguous vertebral bodies or posteriorly between consecutive transverse processes, laminae or other posterior aspects of the vertebrae.

An osseous bridge, or fusion mass, is biologically produced by the body upon skeletal injury. This normal bone healing response is used by surgeons to induce fusion across abnormal spinal segments by recreating spinal injury conditions along the fusion site and then allowing the bone to heal. A successful fusion requires the presence of osteogenic or osteopotential cells, adequate blood supply, sufficient inflammatory response, and appropriate preparation of local bone. This biological environment is typically provided in a surgical setting by decortication, or removal of the outer, cortical bone to expose the vascular, cancellous bone, and the deposition of an adequate quantity of high quality graft material.

A fusion or arthrodesis procedure is often performed to treat an anomaly involving an intervertebral disc. Intervertebral discs, located between the endplates of adjacent vertebrae, stabilize the spine, distribute forces between vertebrae and cushion vertebral bodies. A normal intervertebral disc includes a semi-gelatinous component, the nucleus pulposus, which is surrounded and confined by an outer, fibrous ring called the annulus fibrosis. In a healthy, undamaged spine, the annulus fibrosis prevents the nucleus pulposus from protruding outside the disc space.

Spinal discs may be displaced or damaged due to trauma, disease or aging. Disruption of the annulus fibrosis allows the nucleus pulposus to protrude into the vertebral canal, a condition commonly referred to as a herniated or ruptured disc. The extruded nucleus pulposus may press on the spinal nerve, which may result in nerve damage, pain, numbness, muscle weakness and paralysis. Intervertebral discs may also deteriorate due to the normal aging process or disease. As a disc dehydrates and hardens, the disc space height will be reduced leading to instability of the spine, decreased mobility and pain.

Sometimes the only relief from the symptoms of these conditions is a discectomy, or surgical removal of a portion or all of an intervertebral disc followed by fusion of the adjacent vertebrae. The removal of the damaged or unhealthy disc will allow the disc space to collapse. Collapse of the disc space can cause instability of the spine, abnormal joint mechanics, premature development of arthritis or nerve damage, in addition to severe pain. Pain relief via discectomy and arthrodesis requires preservation of the disc space and eventual fusion of the affected motion segments.

Bone grafts are often used to fill the intervertebral space to prevent disc space collapse and promote fusion of the adjacent vertebrae across the disc space. In early techniques, bone material was simply disposed between the adjacent vertebrae, typically at the posterior aspect of the vertebrae, and the spinal column was stabilized by way of a plate or rod spanning the affected vertebrae. Once fusion occurred the hardware used to maintain the stability of the segment became superfluous and was a permanent foreign body. Moreover, the surgical procedures necessary to implant a rod or plate to stabilize the level during fusion were frequently lengthy and involved.

It was therefore determined that a more optimal solution to the stabilization of an excised disc space is to fuse the vertebrae between their respective end plates, preferably without the need for anterior or posterior plating. There have been an extensive number of attempts to develop an acceptable intra-discal implant that could be used to replace a damaged disc and maintain the stability of the disc interspace between the adjacent vertebrae, at least until complete arthrodesis is achieved. To be successful the implant must provide temporary support and allow bone in growth. Success of the discectomy and fusion procedure requires the development of a contiguous growth of bone to create a solid mass because the implant may not withstand the cyclic compressive spinal loads for the life of the patient.

Many attempts to restore the intervertebral disc space after removal of the disc have relied on metal devices. U.S. Pat. No. 4,878,915 to Brantigan teaches a solid metal plug. U.S. Pat. Nos. 5,044,104; 5,026,373 and 4,961,740 to Ray; 5,015,247 to Michelson and U.S. Pat. No. 4,820,305 to Harms et al., U.S. Pat. No. 5,147,402 to Bohler et al. and U.S. Pat. No. 5,192,327 to Brantigan teach hollow metal cage structures.

Unfortunately, due to the stiffness of the material, some metal implants may stress shield the bone graft, increasing the time required for fusion or causing the bone graft to resorb inside the cage. Subsidence, or sinking of the device into bone, may also occur when metal implants are implanted between vertebrae if fusion is delayed. Metal devices are also foreign bodies which can never be fully incorporated into the fusion mass.

Various bone grafts and bone graft substitutes have also been used to promote osteogenesis and to avoid the disadvantages of metal implants. Autograft is often preferred because it is osteoinductive. Both allograft and autograft are biological materials which are replaced over time with the patient's own bone, via the process of creeping substitution. Over time a bone graft virtually disappears unlike a metal implant which persists long after its useful life. Stress shielding is avoided because bone grafts have a similar modulus of elasticity as the surrounding bone. Commonly used implant materials have stiffness values far in excess of both cortical and cancellous bone. Titanium alloy has a stiffness value of 114 Gpa and 316L stainless steel has a stiffness of 193 Gpa. Cortical bone, on the other hand, has a stiffness value of about 17 Gpa. Moreover, bone as an implant also allows excellent postoperative imaging because it does not cause scattering like metallic implants on CT or MRI imaging.

Various implants have been constructed from bone or graft substitute materials to fill the intervertebral space after the removal of the disc. For example, the Cloward dowel is a circular graft made by drilling an allogenic or autogenic plug from the illium. Cloward dowels are bicortical, having porous cancellous bone between two cortical surfaces. Such dowels have relatively poor biomechanical properties, in particular a low compressive strength. Therefore, the Cloward dowel is not suitable as an intervertebral spacer without internal fixation due to the risk of collapsing prior to fusion under the intense cyclic loads of the spine.

Bone dowels having greater biomechanical properties have been produced and marketed by the University of Florida Tissue Bank, Inc., 1 Progress Boulevard, P.O. Box 31, S. Wing, Alachua, Fla. 32615. Unicortical dowels from allogenic femoral or tibial condyles are available. The University of Florida has also developed a diaphysial cortical dowel having superior mechanical properties. This dowel also provides the further advantage of having a naturally preformed cavity formed by the existing meduallary canal of the donor long bone. The cavity can be packed with osteogenic materials such as bone or bioceramic.

Unfortunately, the use of bone grafts presents several disadvantages. Autograft is available in only limited quantities. The additional surgery also increases the risk of infection and blood loss and may reduce structural integrity at the donor site. Furthermore, some patients complain that the graft harvesting surgery causes more short-term and long-term pain than the fusion surgery.

Allograft material, which is obtained from donors of the same species, is more readily obtained. However, allogenic bone does not have the osteoinductive potential of autogenous bone and therefore may provide only temporary support. The slow rate of fusion using allografted bone can lead to collapse of the disc space before fusion is accomplished.

Both allograft and autograft present additional difficulties. Graft alone may not provide the stability required to withstand spinal loads. Internal fixation can address this problem but presents its own disadvantages such as the need for more complex surgery as well as the disadvantages of metal fixation devices. Also, the surgeon is often required to repeatedly trim the graft material to obtain the correct size to fill and stabilize the disc space. This trial and error approach increases the length of time required for surgery. Furthermore, the graft material usually has a smooth surface which does not provide a good friction fit between the adjacent vertebrae. Slippage of the graft may cause neural and vascular injury, as well as collapse of the disc space. Even where slippage does not occur, micromotion at the graft/fusion-site interface may disrupt the healing process that is required for fusion.

Several attempts have been made to develop a bone graft substitute which avoids the disadvantages of metal implants and bone grafts while capturing advantages of both. For example Unilab, Inc. markets various spinal implants composed of hydroxyapatite and bovine collagen. In each case developing an implant having the biomechanical properties of metal and the biological properties of bone without the disadvantages of either has been extremely difficult or impossible.

A need has remained for fusion spacers which stimulate bone ingrowth and avoid the disadvantages of metal implants yet provide sufficient strength to support the vertebral column until the adjacent vertebrae are fused.

### SUMMARY OF THE INVENTION

In accordance with one aspect of the invention, spinal spacers and compositions are provided for fusion of a motion segment. The spacers include a load bearing member sized for engagement within a space between adjacent vertebrae to maintain the space and an effective amount of an osteogenic composition to stimulate osteoinduction. The osteogenic composition includes a substantially pure osteogenic factor in a pharmaceutically acceptable carrier. In one embodiment the load bearing member includes a bone graft impregnated with an osteogenic composition. In another embodiment, the osteogenic composition is packed within a chamber defined in the graft. The grafts include bone dowels, D-shaped spacers and cortical rings.

In accordance with another aspect of the invention, spinal spacers and compositions are provided for fusion of a motion segment. Spacers include a load bearing body sized for engagement within the space between adjacent vertebrae after discectomy to maintain the space. The body is formed of a bone composition and includes a first end defining a first surface, an opposite second end defining a second surface, a superior face defining a superior vertebral engaging surface and an inferior face defining an inferior vertebral engaging surface. The spacers include means for resisting migration. In one embodiment, the means include a set of migration resistant grooves defined in at least one of the vertebral engaging surfaces. Each of the grooves includes a first face defining an angle of no more than about 90° relative to the engaging surface and a second opposing sloped face. The first and second faces define a pocket therebetween for trapping vertebral bone. In another embodiment the set of grooves is defined in the first portion of the engaging surface and a second set of migration resistant grooves is defined in a second portion of the surface to resist migration in two directions.

An object of the invention, therefore, is to provide spacers for engagement between vertebrae which resist migration of the implanted spacers, yet encourage bone ingrowth and avoid stress shielding. Another benefit of this invention is that it allows the use of bone grafts without the need for metal cages or internal fixation, due to the compressive strength of the spacer and the means for resisting migration.

Another object of the invention is to provide spacers for engagement between vertebrae which encourages bone ingrowth and avoids stress shielding. Another object of the invention is to provide a spacer which restores the intervertebral disc space and supports the vertebral column while promoting bone ingrowth.

One benefit of the spacers of the present invention is that they combine the advantages of bone grafts with the advantages of metals, without the corresponding disadvantages. An additional benefit is that the invention provides a stable scaffold for bone ingrowth before fusion occurs. Still another benefit of this invention is that it allows the use of bone grafts without the need for metal cages or internal fixation, due to the increased speed of fusion. Other objects and further benefits of the present invention will become apparent to persons of ordinary skill in the art from the following written description and accompanying Figures.

### BRIEF DESCRIPTION OF THE DRAWINGS

FIG. 1 is a top perspective view of a bone-dowel according to this invention.

FIG. 2 shows bilateral dowel placement between L5 and the sacrum.

FIG. 3 is a perspective view of a cortical dowel having a chamber.

FIG. 4 is a side perspective view of a dowel according to this invention.

FIG. 5 is a cross-section of another dowel of this invention.

FIG. 6 is a side elevational view of the dowel shown in FIG. 5.

FIG. 7 is a side elevational view of another dowel provided by this invention.

FIG. 8 is a detail of the threads of the dowel shown in FIG. 7.

FIG. 9 is a partial cross-section of a spine showing bilateral placement of two dowels.

FIG. 10 is a partial cross-section of a spine with a cortical ring implanted.

FIG. 11 is a cortical ring packed with an osteogenic material.

FIG. 12 is yet another cortical ring embodiment provided by this invention.

FIG. 13 is another embodiment of a cortical ring provided by this invention.

FIG. 14 is a D-shaped spacer of this invention.

FIG. 15 is a front perspective view of the spacer of FIG. 14:

FIG. 16 is a front elevational view of the spacer depicted in FIG. 14.

FIG. 17 is a top perspective view of the spacer of FIG. 14 showing the chamber packed with a collagen sponge.

FIG. 18 is a top elevational view of a collagen sponge.

FIG. 19 is a D-spaced spacer of this invention having a tool engaging hole.

FIG. 20 is a front elevational view of the spacer FIG. 19.

FIG. 21 is top elevational view of another embodiment of the spacer.

FIG. 22 is a top elevational view of another embodiment of the spacer.

FIG. 23 is a top perspective view of another embodiment of the spacers of this invention having teeth.

FIG. 24 is a top elevational view of another embodiment of the spacer having blades.

FIG. 25 is a front elevational view of the spacer of FIG. 24.

FIG. 26 is a side elevational view of an autograft crock dowel.

FIG. 27 is a side elevational view of an autograft tricortical dowel.

FIG. 28 is a side elevational view of an autograft button dowel.

FIG. 29 is a side elevational view of a hybrid autograft button/allograft crock dowel.

FIG. 30 is a perspective view of a threaded cortical threaded diaphysial dowel having an osteogenic composition packed in the chamber.

FIG. 31 is a side perspective view of a dowel with an osteogenic composition packed within the chamber.

FIG. 32 is a side perspective view of a dowel with a ceramic carrier packed within the chamber.

FIG. 33 is a top elevational view of a spacer having migration resistance grooves.

FIG. 34 is a front elevational view of the spacer of FIG. 33.

FIG. 35 is a side elevational view of the spacer of FIG. 33.

FIG. 36 is a side elevational detailed view of the surface of the spacer of FIG. 33.

FIG. 37 is a side elevational detailed view of the surface of another spacer of this invention.

FIG. 38 is a top elevational view of another embodiment of the spacer having two sets of migration resistance grooves.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated spacers, and such further applications of the principles of the invention as illustrated therein being contemplated as would normally occur to one skilled in the art to which the invention relates.

The present invention provides spacers for engagement between vertebrae which are sized and configured to fill the space left after discectomy. The inventive spacers restore the height of the intervertebral disk space and provide immediate load bearing capability and support for the vertebral column without internal fixation. This invention eliminates the need for invasive autograft harvesting and trial and error trimming of graft material to fit the intra-distal space. The implants advantageously have an anatomically friendly shape and features which increase stability and decrease the risk of complications. In preferred embodiments, the spacers have the compressive strength of cortical bone with the advantage of incorporation of the spacer material without stress shielding. The migration resistance means prevents slippage, expulsion or micromotion. In this way, the spacers of this invention stimulate bone ingrowth like a bone graft and provide sufficient strength to support the vertebral column but avoid the disadvantages of both bone graft and metal implants such as graft migration, stress shielding and the presence of a permanent foreign body.

The migration resistance means increase post-operative stability of the spacer by engaging the adjacent vertebral endplates and anchoring the spacer to prevent expulsion. Such surface features also stabilize the bone-spacer interface and reduce micromotion to facilitate incorporation and fusion. These features also provide increased surface area which facilitates the process of bone healing and creeping substitution for replacement of the donor bone material and fusion.

The present invention also provides bone grafts in synergistic combination with an osteogenic material, such as a bone morphogenic protein (BMP). The combination of BMP with a bone graft provides the advantages of a bone graft while enhancing bone growth into and incorporation of the graft, resulting in fusion quicker than with graft alone. The quicker fusion rates provided by this invention compensate for the less desirable biomechanical properties of graft and makes the use of internal fixation and metal interbody fusion devices unnecessary. The spacers of this invention are not required to support the cyclic loads of the spine for very long because of the quick fusion rates which reduce the biomechanical demands on the spacer. Therefore this invention capitalizes on the advantages of graft while avoiding the disadvantages.

The spinal spacers of this invention include a load bearing member sized for engagement within a space between adjacent vertebrae to maintain the space. The load bearing member is a bone graft in synergistic combination with an osteogenic material. The bone graft is any suitable bone material, preferably of human origin, including tibial, Tibial, humeral, iliac, etc. The load bearing members of this invention include flat D-shaped spacers, bone dowels, cortical rings and any suitably shaped load bearing member composed of bone. A preferred load bearing member is obtained from the diaphysis of a long bone having a medullary canal which forms a natural chamber in the graft.

This invention provides the further advantage of exploiting the discovery that bone is an excellent carrier for osteogenic factors such as bone morphogenic proteins. Hydroxyapatite which is very similar in chemical composition to the mineral in cortical bone is an osteogenic factor-binding agent which controls the rate of delivery of certain proteins to the fusion site. Calcium phosphate compositions such as hydroxyapatite are thought to bind bone morphogenic proteins and prevent BMP from prematurely dissipating from the spacer before fusion can occur. It is further believed that retention of the BMP by the agent permits the protein to initiate the transformation of mesenchymal stem cells into bone producing cells (osteoblasts) within the device at a rate that is conducive to complete and rapid bone formation and ultimately, fusion across the disc space. The spacers of this invention have the advantage of including a load bearing member composed of bone which naturally binds and provides controlled delivery of osteogenic factors such as bone morphogenic proteins.

This invention also capitalizes on the discovery that cortical bone, like metal, can be conveniently machined into the various shapes disclosed herein. In some embodiments, the load bearing members define threads on an outer surface. Machined surfaces, such as threads, provide several advantages that were previously only available with metal implants. Threads allow better control of spacer insertion than can be obtained with a smooth surface. This allows the surgeon to more accurately position the spacer which is extremely important around the critical neurological and vascular structures of the spinal column. Threads and the like also provide increased surface area which facilitates the process of bone healing and creeping substitution for replacement of the donor bone material and fusion. These features also increase post-operative stability of the spacer by engaging the adjacent vertebral endplates and anchoring the spacer to prevent expulsion. This is a major advantage over smooth grafts. Surface features also stabilize the bone-spacer interface and reduce micromotion to facilitate incorporation and fusion.

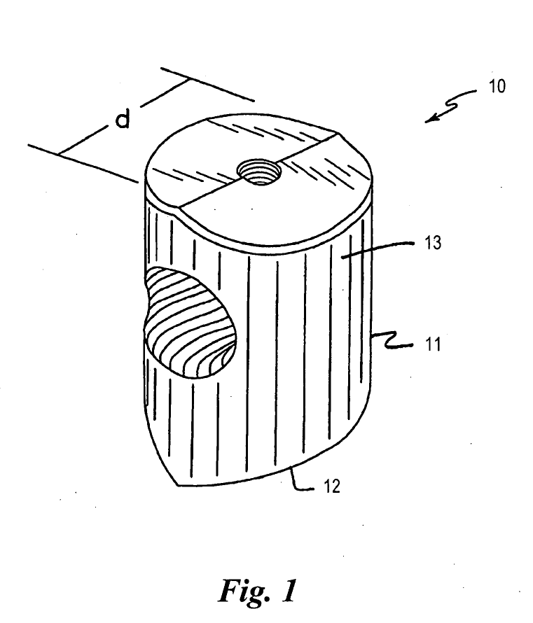

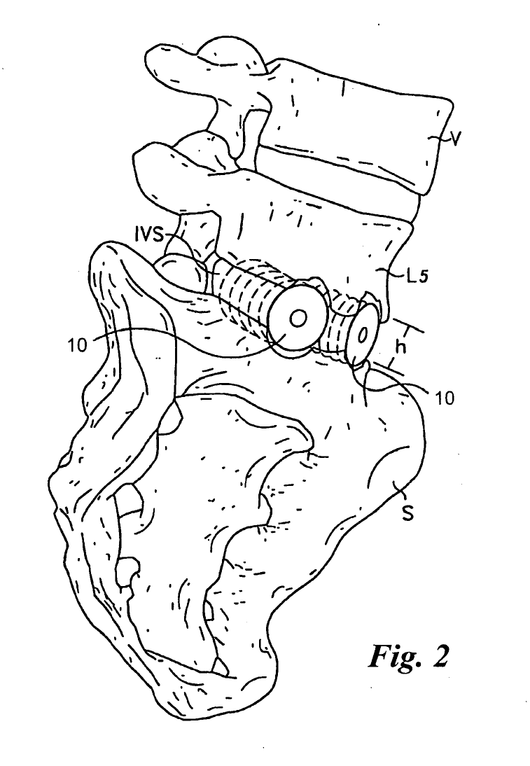

In one specific embodiment depicted in FIG. 1, the load bearing member of the spacer 10 is a bone dowel 11 soaked with an effective amount of an osteogenic composition to stimulate osteoinduction. Preferably, the osteogenic composition includes a substantially pure osteogenic factor in a pharmaceutically acceptable carrier. The dowel 10 includes a wall 12 sized for engagement within the intervertebral space IVS to maintain the space IVS. The wall 12 defines an outer engaging surface 13 for contacting the adjacent vertebrae. The wall 12 is preferably cylindrically so that the bone dowel 10 has a diameter d which is larger than the height h of the space IVS between adjacent vertebrae V or the height of the space between the lowest lumbar vertebrae L5 and the sacrum S as depicted in FIG. 2.

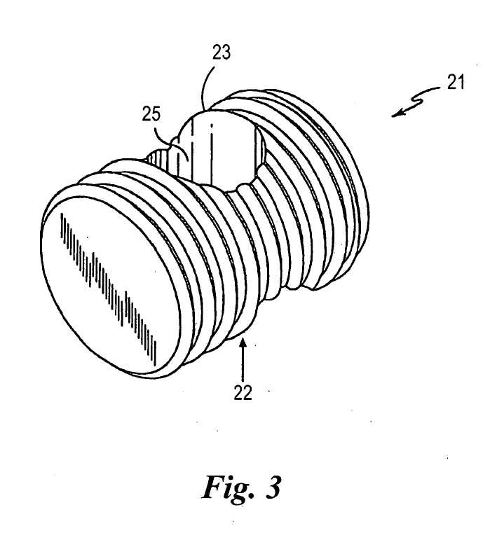

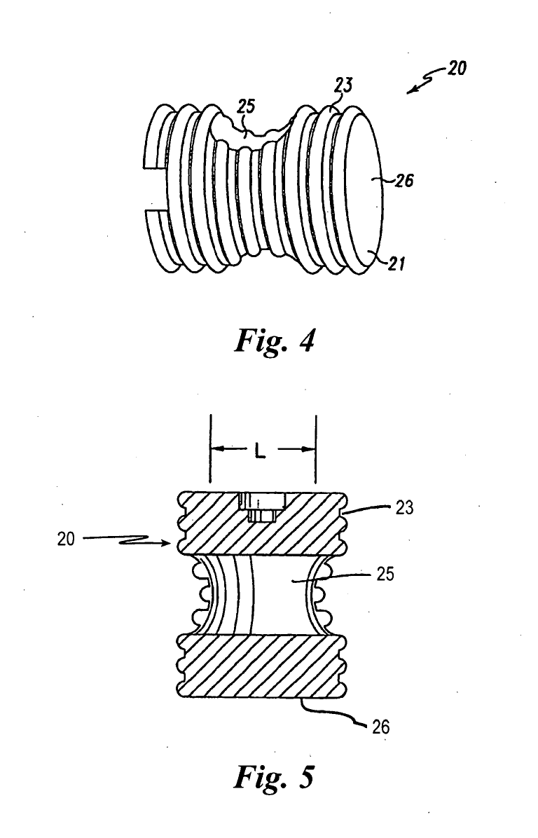

In another embodiment 20 depicted in FIG. 3, the load bearing member is a bone dowel 21 which includes a wall 22 having an engagement surface 23. The wall 22 defines a chamber 25 therethrough. Preferably, the load bearing member is a bone graft obtained from the diaphysis of a long bone having a medullary canal which forms the chamber 25. The chamber 25 is most preferably packed with an osteogenic composition to stimulate osteoinduction. The chamber 25 is preferably defined through a pair of outer engaging surfaces 23 so that the composition has maximum contact with the endplates of the adjacent vertebrae. Referring now to FIG. 4, the spacer 21 includes a solid protective wall 26 which is positionable to protect the spinal cord from escape or leakage of the osteogenic composition 30 within the chamber 25. In anterior approaches, the protective wall 26 is posterior. Preferably, the osteogenic composition 30 has a length which is greater than the length of the chamber (FIGS. 5 and 6) and the composition 30 is disposed within the chamber 25 to contact the end plates of adjacent vertebrae when the spacer 20 is implanted between the vertebrae. This provides better contact of the composition with the end plates to stimulate osteoinduction.

Various features can be machined on the outer surfaces of the dowels of this invention. In one embodiment shown in FIG. 7, the dowel 40 includes an outer engaging surface 41 defining threads 42. The initial or starter thread 47 is adjacent the protective wall 26′. As shown more clearly in FIG. 8, the threads are preferably uniformally machined threads which include teeth 43 having a crest 44 between a leading flank 45 and an opposite trailing flank 46. Preferably the crest 44 of each tooth 43 is flat. In one specific embodiment, the crest 44 of each tooth 43 has a width w of between about 0.020 inches [0.5 mm] and about 0.030 inches [0.66 mm]. The threads 42 preferably define an angle α between the leading flank 45 and the trailing flank 46 of adjacent ones of said teeth 43. The angle a is preferably between about 50 degrees and 70 degrees. Each tooth 43 preferably has a height h′ which is about 0.030 inches [0.66 mm] and about 0.045 inches [1.125 mm].

Referring again to FIG. 7, in some embodiments, the dowel 40 is provided with a tool engaging hole 49 in a wall 48 opposite the solid protective wall 26′. The tool engaging hole 49 is provided in a surface of the dowel which is adjacent the surgeon and opposite the initial thread 47. For an anterior procedure, the tool engaging tool hole 49 would be provided in the anterior surface 48 of the dowel 40. Other machined features are contemplated in the outer or bone engaging surfaces 41. Such machine features include surface roughenings such as knurlings and ratchetings.

In a most preferred embodiment, the tool engaging hole 49 is threaded to receive a threaded tip of an implanting tool.

The spacers of this invention can be inserted using conventional techniques. In accordance with additional aspects of the present invention, methods for implanting an interbody fusion spacer, such as the spacer 40, are contemplated. These methods are also disclosed in commonly assigned, co-pending U.S. patent application Ser. No. 08/804,674, METHODS AND INSTRUMENTS FOR INTERBODY FUSION.

The spacers of this invention can also be inserted using laproscopic technology as described in Sofamor Danek USA's Laproscopic Bone Dowel Surgical Technique, © 1995, 1800 Pyramid Place, Memphis, Tenn. 38132, 1-800-933-2635. Devices of this invention can be conveniently incorporated into Sofamor Danek's laproscopic bone dowel system that facilitates anterior interbody fusions with an approach that is much less-surgical morbid than the standard open anterior retroperitoneal approaches. This system includes templates, trephines, dilators, reamers, ports and other devices required for laproscopic dowel insertion.

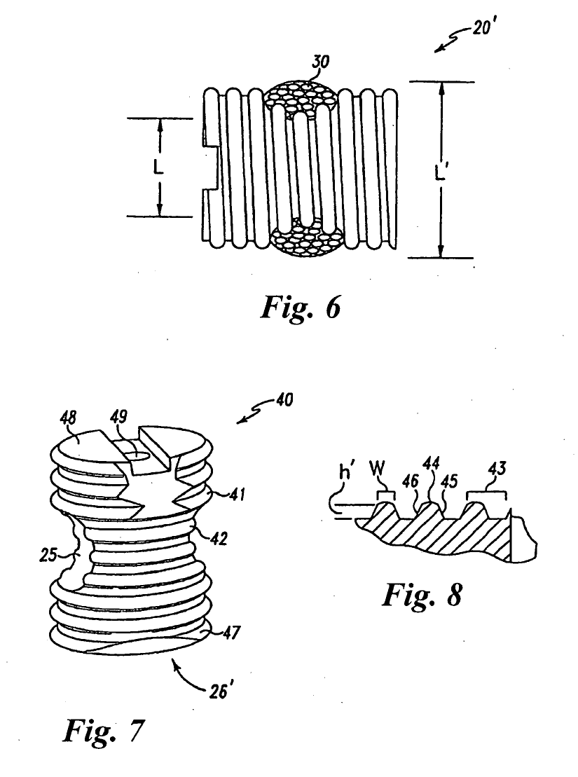

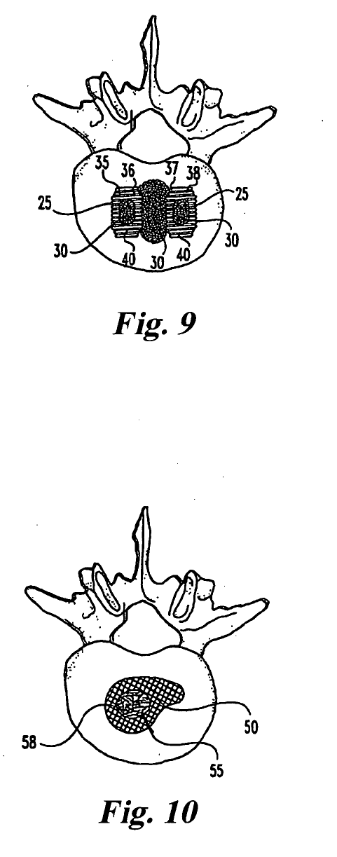

Bilateral placement of dowels 40 is preferred as shown in FIGS. 2 and 9. This configuration provides a substantial quantity of bone graft available for the fusion. The dual bilateral cortical dowels 40 result in a significant area of cortical bone for load bearing and long-term incorporation via creeping substitution, while giving substantial area for placement of osteogenic autogenous bone and boney bridging across the disc space. Comparing FIG. 9 to FIG. 10, it can be seen that bilateral placement of dowels 40 provides a greater surface area of bone material than a single ring allograft 50 which provides only a single chamber 55 for packing with osteogenic material 30. The dual dowel placement results in two chambers 25 that can be filled with an osteogenic composition. Additionally, osteogenic material 30 such as cancellous bone or BMP in a biodegradable carrier may be packed around the dowels. This provides for the placement of a significant amount of osteogenic material as well as four columns 35, 36, 37, 38 of cortical bone for load bearing.

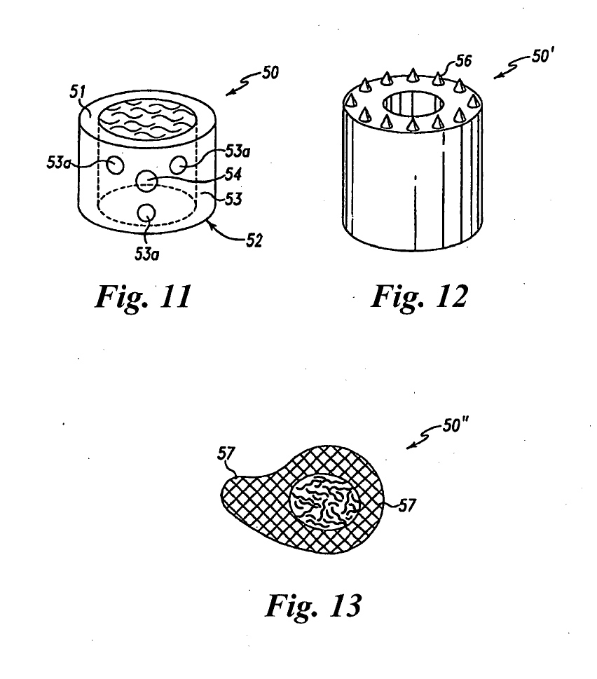

The load bearing member may also include other grafts such as cortical rings as shown in FIG. 11. Such cortical rings 50 are obtained by a cross-sectional slice of the diaphysis of a long bone and include superior surface 51 and inferior surface 52. The graft shown in FIG. 11 includes an outer surface 53 which is adjacent and between the superior 51 and inferior 52 surfaces. In one embodiment bone growth thru-holes 53a are defined through the outer surface 53 to facilitate fusion. The holes 53a allows mesenchymal stem cells to creep in and BMP protein to diffuse out of the graft. This facilitates bone graft incorporation and possibly accelerates fusion by forming anterior and lateral bone bridging outside and through the device. In another embodiment the outer surface 53 defines a tool engaging hole 54 for receiving an implanting tool. In a preferred embodiment, at least one of the superior and/or inferior surfaces 51, 52 are roughened for gripping the end plates of the adjacent vertebrae. The surface roughenings may include teeth 56 on ring 50′ as shown in FIG. 12 or waffle pattern 57 as shown on ring 50″ in FIG. 13. When cortical rings are used as the graft material the ring 50 may be trimmed for a more uniform geometry as shown in FIG. 11 or left in place as shown in FIG. 13.

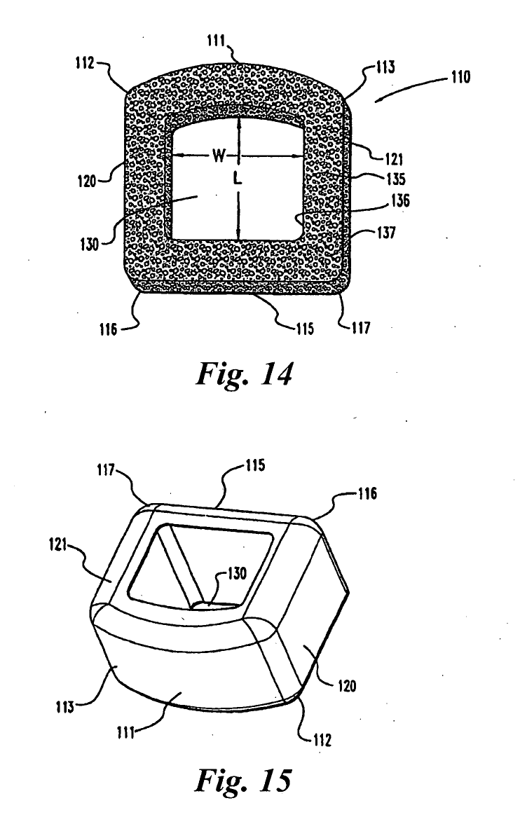

In another specific embodiment, spacers are provided for engagement between vertebrae as depicted in FIGS. 14-16. Spacers of this invention can be conveniently incorporated into current surgical procedures such as, the Smith-Robinson technique for cervical fusion (Smith, M.D., G. W. and R. A. Robinson, M.D., “The Treatment of Certain Cervical-Spine Disorders by Anterior Removal of the Intervertebral Disc and Interbody Fusion”, J. Bone And Joint Surgery, 40-A:607-624 (1958) and Cloward, M.D., R. B., “The Anterior Approach For Removal Of Ruptured Cervical Disks”, in meeting of the Harvey Cushing Society, Washington, D.C., Apr. 22, 1958). In such procedures, the surgeon prepares the endplates of the adjacent vertebral bodies to accept a graft after the disc has been removed. The endplates are generally prepared to be parallel surfaces with a high speed burr. The surgeon then typically sculpts the graft to fit tightly between the bone surfaces so that the graft is held by compression between the vertebral bodies. The bone graft is intended to provide structural support and promote bone ingrowth to achieve a solid fusion of the affected joint. The spacers of this invention avoid the need for this graft sculpting as spacers of known size and dimensions are provided. This invention also avoids the need for a donor surgery because the osteoinductive properties of autograft are not required. The spacers can be combined with osteoinductive materials that make allograft osteoinductive. Therefore, the spacers of this invention speed the patient's recovery by reducing surgical time, avoiding a painful donor surgery and inducing quicker fusion.

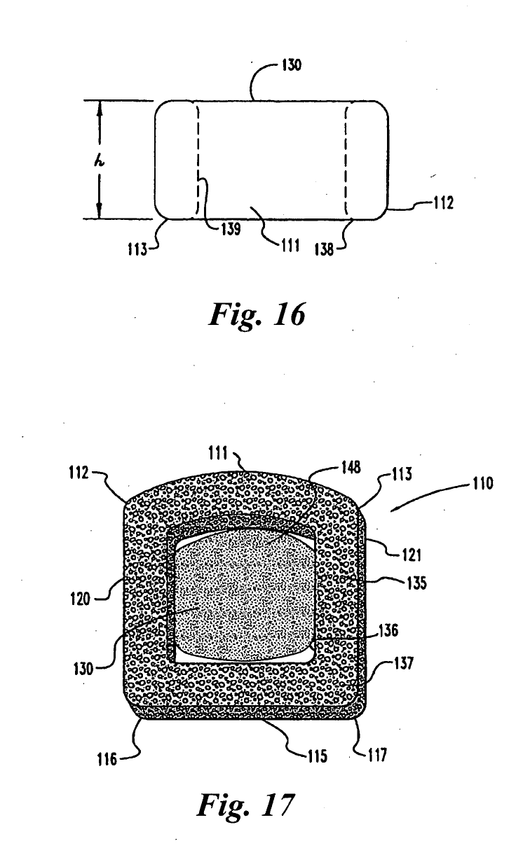

The spacer 110 includes an anterior wall 111 having opposite ends 112, 113, a posterior wall 115 having opposite ends 116, 117 and two lateral walls 120, 121. Each of the lateral walls 120, 121 is connected between the opposite ends 112, 113, 116, 117 of the anterior 111 and posterior 115 walls to define a chamber 130. The walls are each composed of bone and also include the superior face 135 which defines a first opening 136 in communication with the chamber 130. The superior face 135 includes a first friction or vertebral engaging surface 137. As shown in FIG. 16, the walls further include an opposite inferior face 138 defining a second opening 139 which is in communication with the chamber 130. The chamber 130 is preferably sized to receive an osteogenic composition to facilitate bone growth. The inferior face 138 includes a second friction or second vertebral engaging surface (not shown) which is similar to or identical to the first friction or vertebral engaging surface 137.

In one specific embodiment for an intervertebral disc replacement spacer, a hollow D-shaped spinal spacer is provided. The anterior wall 111 as shown in FIGS. 14-16 is convexly curved. This anterior curvature is preferred to conform to the geometry of the adjacent vertebral bone and specifically to the harder cortical bone of the vertebrae. The D-shape of the spacer 110 also prevents projection of the anterior wall 111 outside the anterior aspect of the disc space, which can be particularly important for spacers implanted in the cervical spine.

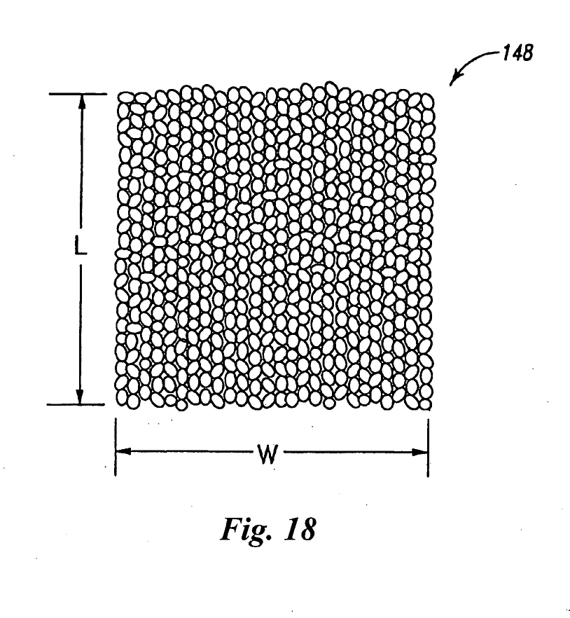

In one specific embodiment shown in FIGS. 17 and 18, the D-shaped spacer 110 includes a collagen sponge 148 having a width w and length 1 which are each slightly greater than the width W and length L of the chamber. In a preferred embodiment, the sponge 148 is soaked with freeze dried rhBMP-2 reconstituted in buffered physiological saline and then compressed into the chamber 130. The sponge 148 is held within the chamber 130 by the compressive forces provided by the sponge 148 against the walls 111, 115, 120, 121 of the spacer 110.

The spacers are shaped advantageously for cervical arthrodesis. The flat posterior and lateral walls 115, 120 and 121, as shown in FIG. 14, can be easily incorporated into Smith Robinson surgical fusion technique. After partial or total discectomy and distraction of the vertebral space, the surgeon prepares the end plates for the spacer 110 preferably to create flat posterior and lateral edges. The spacer 110 fits snugly with its flat surfaces against the posterior and lateral edges which prevents medial and lateral motion of the spacer 110 into vertebral arteries and nerves. This also advantageously reduces the time required for the surgery by eliminating the trial and error approach to achieving a good fit with bone grafts because the spacers can be provided in predetermined sizes.

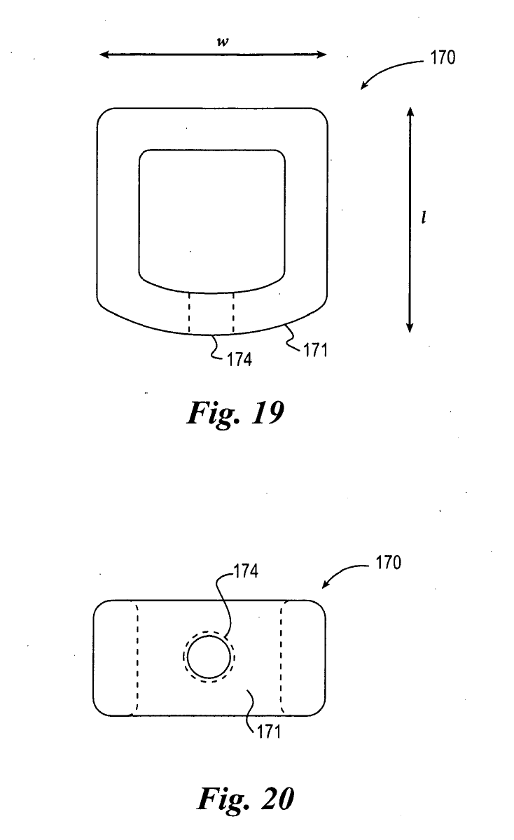

According to another specific embodiment depicted in FIGS. 19 and 20, the spacer 170 includes an anterior wall 171 defining a tool engaging hole 174. In a most preferred embodiment, the tool engaging hole 174 is threaded for receiving a threaded implanting tool.

In the preferred embodiments, the spacers are provided with migration resistance means.

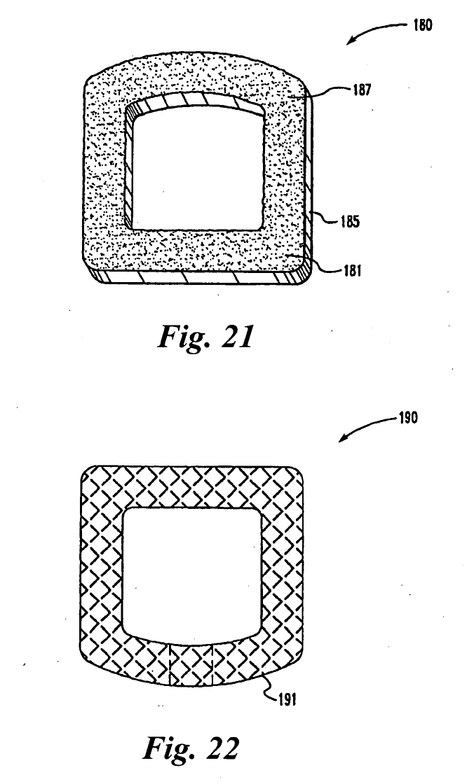

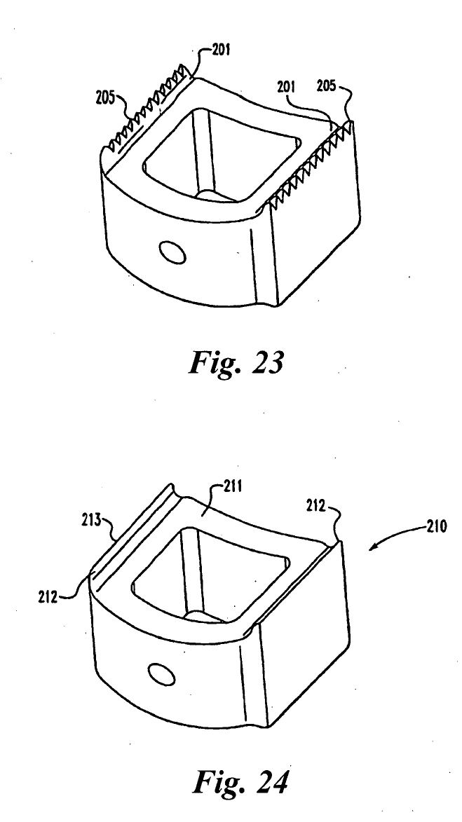

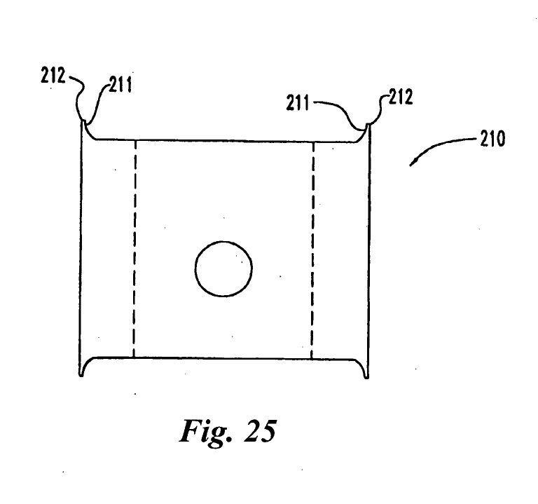

The engaging surfaces of the spacers are machined to facilitate engagement with the endplates of the vertebrae and prevent slippage of the spacer as is sometimes seen with smooth graft prepared, at the time of surgery. The spacer 180 may be provided with a roughened surface 181 on one of the engaging surfaces 187 of one or both of the superior face 185 or inferior face (not shown) as shown in FIG. 21. The roughened surface 191 of the spacer 190 may include a waffle or other suitable pattern as depicted in FIG. 22. In one preferred embodiment shown in FIG. 23, the engaging surfaces 201 include teeth 205 which provide biting engagement with the endplates of the vertebrae. In another embodiment (FIGS. 24 and 25), the spacer 210 includes engaging surfaces 211 machined to include one or more blades 212. Each blade includes a cutting edge 213 configured to pierce a vertebral end-plate. The blade 212 can be driven into the bone surface to increase the initial stability of the spacer.

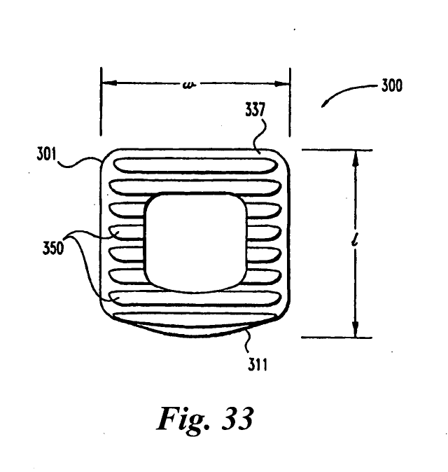

In a preferred embodiment depicted in FIGS. 33-36, the migration resistance means includes a set of expulsion resistance grooves defined in the body 301 of the spacer 300. In this spacer, the superior and inferior vertebral engaging surfaces 337 and 340 define a set of migration resistance grooves 350. As shown more clearly in FIG. 36 each of the grooves 350 includes a first face 355. The first face 355 defines an angle α₁ no more than about 90° relative to the engaging surface 337. Preferably, the angle α₁ is 90°. In other words, the first face 355 is preferably perpendicular to the engaging surface 337. Each groove 350 also includes a second, opposing and sloped-face 360. The sloped face 360 preferably forms an angle α₂ relative to a line 1 which is parallel to the first face 355. The first face 355 and second face 360 define a pocket 370 therebetween for trapping vertebral bone.

Preferably each of the grooves 350 of the set 302 are arranged in series in that each second face 360 slants in the same direction as the others. In the embodiment shown in FIGS. 33-36, each of the grooves 350 slants away from the posterior or second end 315 and towards the first end or anterior wall 311 of the body 301. In this embodiment the engaging surface 337 defines a peak 375 between each of the grooves 350. The peak 375 preferably defines a flattened surface. The vertebral engaging surface 337 may be provided with a cutting edge 380 between the first face 355 and the engaging surface 375.

Referring now to the spacer 400 of FIG. 37, the exact configuration of the grooves may vary. For example, the first face 355 may have a first height h₁ between the pocket 470 and the engaging surface 437 which is taller than a second height h₂ of the second face 460. In this embodiment, the peak 475 is sloped toward the cutting edge 480.

In preferred embodiments, the pocket 370 is substantially arcuate or circular in shape. The pocket is configured for collecting and trapping vertebral bone if the spacer migrates after it is implanted. For example, the embodiment depicted in FIGS. 33-36 has grooves that resist migration in the direction of the arrow A. If the spacer is implanted with the first or anterior end 311 to the anterior of the patient using an anterior approach, the anterior tissues will be weakened and migration will most likely occur in the anatomically anterior direction. The spacer can be configured for implantation with the grooves facing in a direction that resists that anterior migration. If a force urges the spacer 300 in the anterior direction, the edge 380 of the peak 375 will dig into the vertebral bone and bone will collect in the pocket 370.

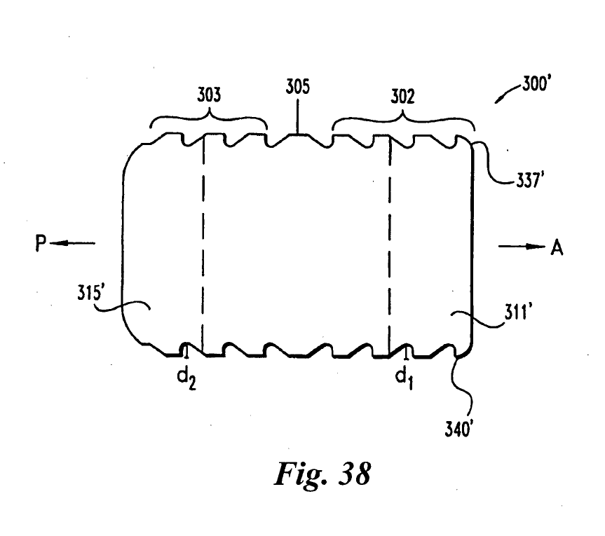

The spacers of this invention may also be provided with means that resist migration in two directions. Referring now to FIG. 38, the spacer 300′ includes a first set of grooves 303 which resist migration in the direction of arrow A and a second set of grooves 302 which resist migration in the direction of arrow P. The two sets of grooves 302 and 303 meet at a flattened bridge member 305. The first set of grooves 302 slants towards the first end 311′ and resists migration in the direction of the arrow A. The second set of grooves 303 slants towards the second end 315′ and resists migration in the direction of the arrow P. In this way the grooves resist micromotion, migration and expulsion.

As shown in FIG. 38, the depth of the grooves may vary between the two sets 302 and 303. The grooves of the two sets 302 and 303 have a depth d₁, d₂ below the vertebral engaging surface 337′ and 340′. The grooves of the first set 302 or the second set 303 may be deeper than the other as needed for the particular application.

The spacers of this invention are preferably formed of a bone composition or material. The bone may be autograft, allograft, xenograft or any of the above prepared in a variety of ways. Cortical bone is preferred for its compressive strength. In one embodiment, the spacers are obtained as a cross sectional slice of a shaft of a long bone. For example, various shaped spacers may be obtained by machining a cortical ring into the desired configuration. The exterior surfaces of the walls can be formed by machining the ring to a D-shape. Material from the medullary canal of the ring can be removed to form a chamber. Surface features and migration resistance means can be defined into the surface of the spacers using conventional machining methods and a standard milling machine which have been adapted to bone. Various methods and procedures are known for treating and processing bone to provide bone materials and compositions. These methods and procedures can be applied to the present invention as long as the resulting bone material provides a sufficient compressive strength for the intended application.

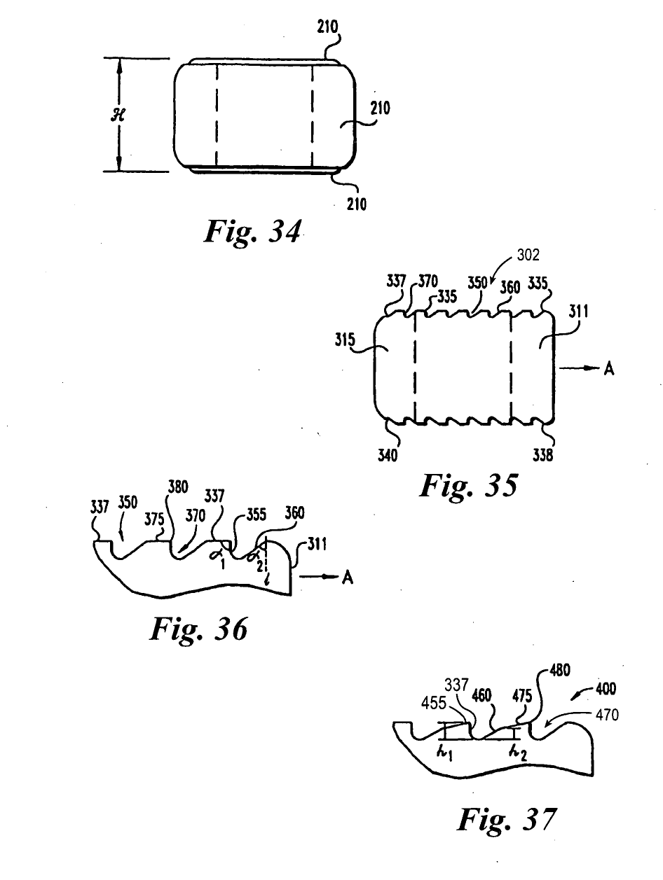

Spacers of the present invention can be made to any suitable size or shape which is suitable for the intended application. Referring now to FIGS. 33 and 34, the spacer has a width W of preferably 11 to 14 millimeters, a length L of preferably between about 11 and 14 millimeters and a height H of about 7 millimeters. The height H is the distance between the highest peak 375 on the superior vertebral engaging surface 337 and the highest peak 375 on the inferior vertebral engaging surface 340.

Advantageously, the intervertebral spacers of the present invention may not require internal fixation. The spacers are contained by the compressive forces of the surrounding ligaments and muscles, and the disc annulus if it has not been completely removed. Temporary external immobilization and support of the instrumented and adjacent vertebral levels, with a cervical collar, lumbar brace or the like, is generally recommended until adequate fusion is achieved.

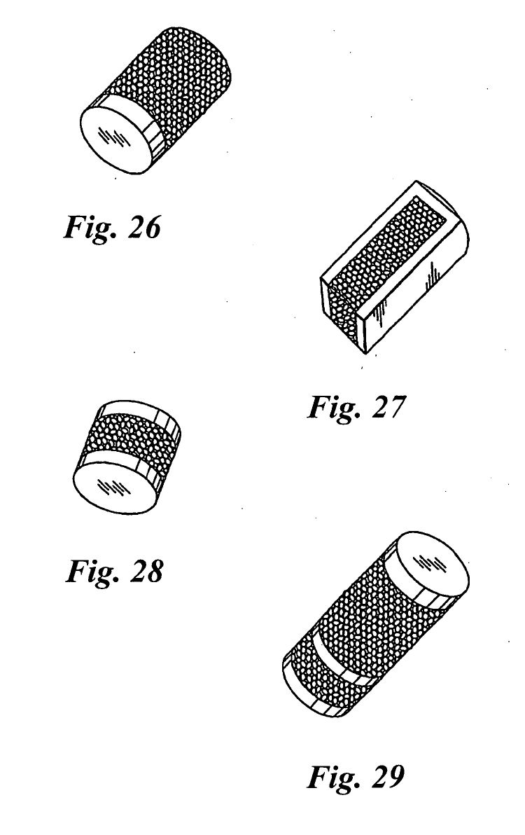

Again, any suitable load bearing member which can be synergistically combined with an osteogenic composition is contemplated. Other potential load bearing members include allograft crock dowels (FIG. 26), tricortical dowels (FIG. 27), button dowels (FIG. 28) and hybrid allograft button-allograft crock dowels (FIG. 29).

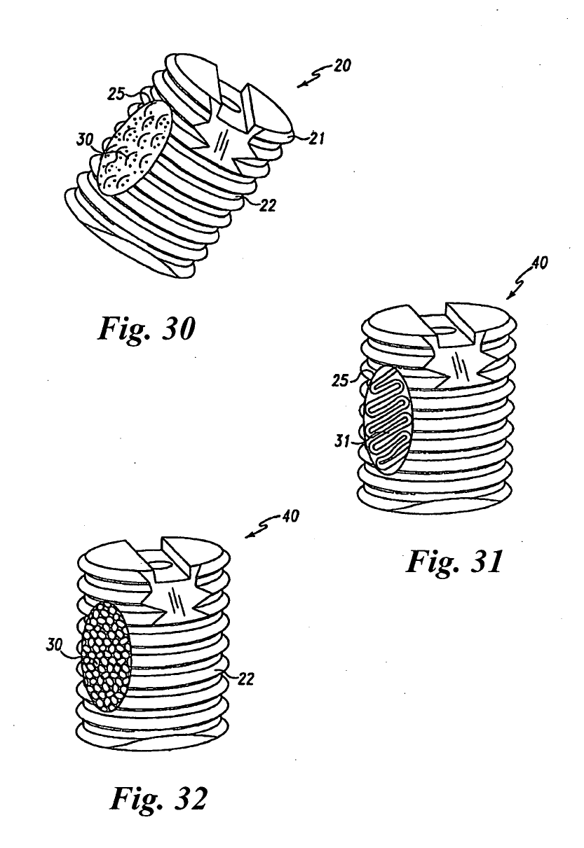

Again, any osteogenic material can be applied to the spacers of this invention by packing the chamber 25,130 with an osteogenic material 30,148 as shown in FIGS. 17 and 30, by impregnating the graft with a solution including an osteogenic composition or by both methods combined. The composition may be applied by the surgeon during surgery or the spacer may be supplied with the composition preapplied. In such cases, the osteogenic composition may be stabilized for transport and storage such as by freeze-drying. The stabilized composition can be rehydrated and/or reactivated with a sterile fluid such as saline or water or with body fluids applied before or after implantation. Any suitable osteogenic material or composition is contemplated, including autograft, allograft, xenograft, demineralized bone, synthetic and natural bone graft substitutes, such as bioceramics and polymers, and osteoinductive factors. The term osteogenic composition used here means virtually any material that promotes bone growth or healing including natural, synthetic and recombinant proteins, hormones and the like.

Autograft can be harvested from locations such as the iliac crest using drills, gouges, curettes and trephines and other tools and methods which are well known to surgeons in this field. Preferably, autograft is harvested from the iliac crest with a minimally invasive donor surgery. The graft may include osteocytes or other bone reamed away by the surgeon while preparing the end plates for the spacer.

Advantageously, where autograft is chosen as the osteogenic material, only a very small amount of bone material is needed to pack the chamber 130. The autograft itself is not required to provide structural support as this is provided by the spacer 110. The donor surgery for such a small amount of bone is less invasive and better tolerated by the patient. There is usually little need for muscle dissection in obtaining such small amounts of bone. The present invention therefore eliminates many of the disadvantages of autograft.

The osteogenic compositions used in this invention preferably comprise a therapeutically effective amount of a substantially pure bone inductive factor such as a bone morphogenetic protein in a pharmaceutically acceptable carrier. The preferred osteoinductive factors are the recombinant human bone morphogenic proteins (rhBMPs) because they are available in unlimited supply and do not transmit infectious diseases. Most preferably, the bone morphogenetic protein is a rhBMP-2, rhBMP-4 or heterodimers thereof. The concentration of rhBMP-2 is generally between about 0.4 mg/ml to about 1.5 mg/ml, preferably near 1.5 mg/ml. However, any bone morphogenetic protein is contemplated including bone morphogenetic proteins designated as BMP-1 through BMP-13. BMPs are available from Genetics Institute, Inc., Cambridge, Mass. and may also be prepared by one skilled in the art as described in U.S. Pat. Nos. 5,187,076 to Wozney et al.; 5,366,875 to Wozney et al.; 4,877,864 to Wang et al.; 5,108,922 to Wang et al.; 5,116,738 to Wang et al.; 5,013,649 to Wang et al.; 5,106,748 to Wozney et al.; and PCT Patent Nos. WO93/00432 to Wozney et al.; WO94/26893 to Celeste et al.; and WO94/26892 to Celeste et al. All osteoinductive factors are contemplated whether obtained as above or isolated from bone. Methods for isolating bone morphogenic protein from bone are described in U.S. Pat. No. 4,294,753 to Urist and Urist et al., 81 PNAS 371, 1984.

The choice of carrier material for the osteogenic composition is based on biocompatibility, biodegradability, mechanical properties and interface properties as well as the structure of the load bearing member. The particular application of the compositions of the invention will define the appropriate formulation. Potential carriers include calcium sulphates, polylactic acids, polyanhydrides, collagen, calcium phosphates, polymeric acrylic esters and demineralized bone. The carrier may be any suitable carrier capable of delivering the proteins. Most preferably, the carrier is capable of being eventually resorbed into the body. One preferred carrier is an absorbable collagen sponge marketed by Integral LifeSciences Corporation under the trade name Helistat® Absorbable Collagen Hemostatic Agent. Another preferred carrier is an open cell polylactic acid polymer (OPLA). Other potential matrices for the compositions may be biodegradable and chemically defined calcium sulfates, calcium phosphates such as tricalcium phosphate (TCP) and hydroxyapatite (HA) and including injectable bicalcium phosphates (BCP), and polyanhydrides. Other potential materials are biodegradable and biologically derived, such as bone or dermal collagen. Further matrices are comprised of pure proteins or extracellular matrix components. The osteoinductive material may also be an admixture of BMP and a polymeric acrylic ester carrier, such as polymethylmethacrylic.

For packing the chambers of the spacers of the present invention, the carriers are preferably provided as a sponge 50,30 which can be compressed into the chamber 55 (FIG. 10) or 25 (FIG. 30) or as strips or sheets which may be folded to conform to the chamber as shown in FIG. 31. Preferably, the carrier has a width and length which are each slightly greater than the width and length of the chamber. In the most preferred embodiments, the carrier is soaked with a rhBMP-2 solution and then compressed into the chamber. As shown in FIG. 30, the sponge 30 is held within the chamber 25 by the compressive forces provided by the sponge 30 against the wall 22 of the dowel 21. It may be preferable for the carrier to extend out of the openings of the chamber to facilitate contact of the osteogenic composition with the highly vascularized tissue surrounding the fusion site. The carrier can also be provided in several strips sized to fit within the chamber. The strips can be placed one against another to fill the interior. As with the folded sheet, the strips can be arranged within the spacer in several orientations. Preferably, the osteogenic material, whether provided in a sponge, a single folded sheet or in several overlapping strips, has a length corresponding to the length and width of the chamber.

The most preferred carrier is a biphasic calcium phosphate ceramic. FIG. 32 shows a ceramic carrier 32 packed within a dowel 40. Hydroxyapatite/tricalcium phosphate ceramics are preferred because of their desirable bioactive properties and degradation rates in vivo. The preferred ratio of hydroxyapatite to tricalcium phosphate is between about 0:100 and about 65:35. Any size or shape ceramic carrier which will fit into the chambers defined in the load bearing member are contemplated. Ceramic blocks are commercially available from Sofamor Danek Group, B. P. 4-62180 Rang-du-Fliers, France and Bioland, 132 Route d:Espagne, 31100 Toulouse, France. Of course, rectangular and other suitable shapes are contemplated. The osteoinductive factor is introduced into the carrier in any suitable manner. For example, the carrier may be soaked in a solution containing the factor.

In a preferred embodiment, an osteogenic composition is provided to the pores of the load bearing member. The bone growth inducing composition can be introduced into the pores in any suitable manner. For example, the composition may be injected into the pores of the graft. In other embodiments, the composition is dripped onto the graft or the graft is soaked in a solution containing an effective amount of the composition to stimulate osteoinduction. In either case the pores are exposed to the composition for a period of time sufficient to allow the liquid to thoroughly soak the graft. The osteogenic factor, preferably a BMP, may be provided in freeze-dried form and reconstituted in a pharmaceutically acceptable liquid or gel carrier such as sterile-water, physiological saline or any other suitable carrier. The carrier may be any suitable medium capable of delivering the proteins to the spacer. Preferably the medium is supplemented with a buffer solution as is known in the art. In one specific embodiment of the invention, rhBMP-2 is suspended or admixed in a carrier, such as water, saline, liquid collagen or injectable BCP. The BMP solution can be dripped into the graft or the graft can be immersed in a suitable quantity of the liquid. In a most preferred embodiment, BMP is applied to the pores of the graft and then lypholized or freeze-dried. The graft-BMP composition can then be frozen for storage and transport.

Advantageously, the intervertebral spacers of the present invention may not require internal fixation. The spacers are contained by the compressive forces of the surrounding ligaments and muscles, and the disc annulus if it has not been completely removed. Temporary external immobilization and support of the instrumented and adjacent vertebral levels, with a cervical collar, lumbar brace or the like, is generally recommended until adequate fusion is achieved.

Although the spacers and compositions of this invention make the use of metal devices typically unnecessary, the invention may be advantageously combined with such devices. The bone graft-osteogenic compositions of the invention can be implanted within any of the various prior art metal cages.

The following specific examples are provided for purposes of illustrating the invention, and no limitations on the invention are intended thereby.

### EXPERIMENTAL I

### Preparation of Devices

### Example 1

### Diaphysial Cortical Bone Dowel

A consenting donor (i.e., donor card or other form of acceptance to serve as a donor) was screened for a wide variety of communicable diseases and pathogens, including human immunodeficiency virus, cytomegalovirus, hepatitis B, hepatitis C and several other pathogens. These tests may be conducted by any of a number of means conventional in the art, including but not limited to ELISA assays, PCR assays, or hemagglutination. Such testing follows the requirements of: (i) American Association of Tissue Banks; Technical Manual for Tissue Banking, Technical Manual—Musculoskeletal Tissues, pages M19-M20; (ii) The Food and Drug Administration, Interim Rule, Federal Register/Vol. 50, No. 238/Tuesday, Dec. 14, 1993/Rules and Regulations/65517, D. Infectious Disease Testing and Donor Screening; (iii) MMWR/Vol. 43/No. RR-8, Guidelines for Preventing Transmission of Human Immunodeficiency Virus Through Transplantation of Human Tissue and Organs, pages 4-7; (iv) Florida Administrative Weekly, Vol. 10, No. 34, Aug. 21, 1992, 59A-1.001-014 59A-1.005(12)(c), F.A.C., (12)(a)-(h), 59A-1.005(15), F.A.C., (4) (a)-(8). In addition to a battery of standard biochemical assays, the donor, or their next of kin, was interviewed to ascertain whether the donor engaged in any of a number of high risk behaviors such as having multiple sexual partners, suffering from hemophilia, engaging in intravenous drug use etc. After the donor was ascertained to be acceptable, the bones useful for obtention of the dowels were recovered and cleaned.

A dowel was obtained as a transverse plug from the diaphysis of a long bone using a diamond tipped cutting bit which was water cleaned and cooled. The bit was commercially available (Starlite, Inc.) and had a generally circular nature and an internal vacant diameter between about 10 mm to about 20 mm. The machine for obtention of endo- and cortical dowels consisted of a pneumatic driven miniature lathe which is fabricated from stainless steel and anodized aluminum. It has a spring loaded carriage which travels parallel to the cutter. The carriage rides on two runners which are 1.0 inch stainless rods and has a travel distance of approximately 8.0 inches. One runner has set pin holes on the running rod which will stop the carriage from moving when the set pin is placed into the desired hole. The carriage 15 moveable from side to side with a knob which has graduations in metric and in English. This allows the graft to be positioned. On this carriage is a vice which clamps the graft and holds it in place while the dowel is being cut. The vice has a cut out area in the jaws to allow clearance for the cutter. The lathe has a drive system which is a pneumatic motor with a valve controller which allows a desired RPM to be set.

First, the carriage is manually pulled back and locked in place with a set pin. Second, the graft is loaded into the vice and is aligned with the cutter. Third, the machine is started and the RPM is set, by using a knob on the valve control. Fourth, the set pin, which allows the graft to be loaded onto the cutter to cut the dowel. Once the cutter has cut all the way through the graft the carriage will stop on a set pin. Fifth, sterile water is used to eject dowel out of the cutter. It is fully autoclavable and has a stainless steel vice and/or clamping fixture to hold grafts for cutting dowels. The graft can be positioned to within 0.001″ of an inch which creates dowel uniformity during the cutting process.

The cutter used in conjunction with the above machine can produce dowels ranging from 5 mm to 30 mm diameters and the sizes of the cutters are 10.6 mm; 11.0 mm; 12.0 mm; 13.0 mm; 14.0 mm; 16.0 mm; and 18.0 mm. The composition of the cutters is stainless steel with a diamond powder cutting surface which produces a very smooth surface on the wall of the dowel's. In addition, sterile water is used to cool and remove debris from graft and/or dowel as the dowel is being cut (hydro infusion). The water travels down through the center of the cutter to irrigate as well as clean the dowel under pressure. In addition, the water aides in ejecting the dowel from the cutter.

The marrow was then removed from the medullary canal of the dowel and the cavity cleaned to create of chamber. The final machined product may be stored, frozen or freeze-dried and vacuum sealed for later use.

### Example 2

### Threaded Dowels

A diaphysial cortical bone dowel is prepared as described above. The plug is then machined, preferably in a class 10 clean room, to the dimensions desired. The machining is preferably conducted on a lathe such as a jeweler's lathe or machining tools may be specifically designed and adapted for this purpose. A hole is then drilled through the anterior wall of the dowel. The hole is then tapped to receive a threaded insertion tool.

### Example 3

### Bone dowel soaked with rhBMP-2

A threaded dowel is obtained through the methods of Examples 1 and 2.

A vial containing 4.0 mg of lyphilized rhBMP-2 (Genetics Institute) is constituted with 1 mL, sterile water (Abbott Laboratories) for injection to obtain a 4.0 mg/mL solution as follows:

1. Using a 3-cc syringe and 22G needle, slowly inject 1.0 mL sterile water for injection into the vial containing lyphilized rhBMP-2.

2. Gently swirl the vial until a clear solution is obtained. Do not shake.

The dilution scheme below is followed to obtain the appropriate rhBMP-2 concentration. This dilution provides sufficient volume for two dowels. The dilutions are performed as follows:

1. Using a 5-cc syringe, transfer 4.0 mL of MFR 906 buffer (Genetics Institute) into a sterile vial.

2. Using a 1-cc syringe, transfer 0.70 mL reconstituted rhBMP-2 into the vial containing the buffer.

3. Gently swirl to mix.

### Dilution Scheme

| INITIAL rhBMP-2 | rhBMP-2 | MFR-842 | FINAL rhBMP-2 |
|---|---|---|---|
| CONCENTRATION | VOLUME | VOLUME | CONCENTRATION |
| (mg/mL) | (mL) | (mL) | (mg/mL) |
| 4.0 | 0.7 | 4.0 | 0.60 |

1. Using a 3-cc syringe and 22G needle, slowly drip 2.0 mL of 0.60 mg/mL rhBMP-2 solution onto the Bone Dowel.

2. Implant immediately.

### Example 4

### Bone Dowel Packed with BMP-2/Collagen Composition

A threaded dowel is obtained through the methods of Examples 1 and 2.

A vial containing 4.0 mg of lyphilized rhBMP-2 (Genetics Institute) is constituted with 1 mL sterile water (Abbott Laboratories) for injection to obtain a 4.0 mg/mL solution as follows:

1. Using a 3-cc syringe and 22G needle, slowly inject 1.0 mL sterile water for injection into the vial containing lyphilized rhBMP-2.

2. Gently swirl the vial until a clear solution is obtained. Do not shake.

The dilution scheme below is followed to obtain the appropriate rhBMP-2 concentration. The dilutions are performed as follows:

1. Using a 3-cc syringe, transfer 2.5 mL of MFR-842 buffer (Genetics Institute) into a sterile vial.

2. Using a 1-cc syringe, transfer 0.30 mL of 4.0 mg/mL reconstituted rhBMP-2 into the vial containing the buffer.

3. Gently swirl to mix.

### Dilution Scheme

| INITIAL rhBMP-2 | rhBMP-2 | MFT-842 | FINAL rhBMP-2 |
|---|---|---|---|
| CONCENTRATION | VOLUME | VOLUME | CONCENTRATION |
| (mg/mL) | (mL) | (mL) | (mg/mL) |
| 4.0 | 0.3 | 2.5 | 0.43 |

The rhBMP-2 solution is applied to a Helistat sponge (Genetics Institute) as follows:

1. Using sterile forceps and scissors, cut a 7.5 cm×2.0 cm strip of Helistat sponge off of a 7.5×10 cm (3″×4″) sponge.

2. Using a 1-cc syringe with a 22-G needle, slowly drip approximately 0.8 mL of 0.43 mg/mL rhBMP-2 solution uniformly onto the Helistat sheet.

3. Using sterile forceps, loosely pack the sponge into the chamber of the dowel.

4. Using a 1-cc syringe with a 22-G needle, inject the remaining 0.8 mL of 0.43 mg/mL rhBMP-2 into the sponge in the dowel through the openings of the chamber.

5. Implant immediately.

### Example 5

### Bone dowel packed rhBMP-2/ha/TCP composition

A threaded dowel is obtained through the methods of Examples 1 and 2. A vial containing 4.0 mg of lyphilized rhBMP-2 (Genetics Institute) is constituted with 1 mL sterile water (Abbott Laboratories) for injection to obtain a 4.0 mg/mL solution as follows:

1. Using a 3-cc syringe and 22G needle, slowly inject 1.0 mL sterile water for injection into the vial containing lyphilized rhBMP-2.

2. Gently swirl the vial until a clear solution is obtained. Do not shake.

A cylindrical block of biphasic hydrozyapatite/tricalcium phosphate (Bioland) is wetted with a 0.4 mg/mL rhBMP-2 solution. The BMP-ceramic block is packed into the chamber of the dowel and the dowel is then implanted.

### Example 6

### Cortical Ring

A screened consenting donor is chosen as described in EXAMPLE 1 as follows. A cortical ring is obtained as a cross-sectional slice of the diaphysis of a human long bone and then prepared using the methods described in Example 1. The ring is packed with an osteogenic composition as described in EXAMPLE 4 or 5.

### Example 7

### Spacers

A screened consenting donor is chosen as described in EXAMPLE 1. A D-shaped cervical spacer is obtained as a cross-sectional slice of a diaphysis of a long bone and then prepared using the methods of Example 1. The exterior surfaces of the walls are formed by machining the slice to a D-shape. The engaging surfaces of the spacer are provided with knurlings by a standard milling machine. A hole is then drilled through the anterior wall of the spacer. The hole is then tapped to engage a threaded insertion tool. The chamber of the spacer is then packed with an osteogenic composition as described in EXAMPLE 4 or 5.

### CONCLUSION

The combination of BMP with a bone graft provides superior results. Quicker fusion rates provide enhanced mechanical strength sooner. Bone is an excellent protein carrier which provides controlled release of BMP to the fusion site. When the bone graft is a threaded cortical dowel, the biomechanical superiority of the load bearing dowel is superbly combined with the enhanced fusion rates of the BMP-bone combination.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

1. A textured bone allograft comprising: a plurality of closely spaced protrusions, each protrusion comprising a triangular shaped cross-section.

2.-66. (canceled)
